(19) United States
(12) Patent Application Publication
(10) Pub. No.: US 2003/0124720 A1
(43) Pub. Date: Jul. 3, 2003

(54) Conditioned culture media

(76) Inventors: Luc Schoonjans, Wilsele (BE); Randall Moreadith, Chapel Hill, NC (US)

Correspondence Address: Barbara E. Johnson, Webb Ziesenheim Logsdon Orkin & Hanson, 700 Koppers Building, 436 Seventh Avenue, Pittsburgh, PA, 15219, US

(21) Appl. No.: 10/038,279

(22) Filed: Jan. 4, 2002

Related U.S. Application Data
(63) Continuation-in-part of application No. 09/894,913, filed on Jun. 28, 2001.
Application No. 09/894,913 is a continuation-in-part of application No. 09/628,883, filed on Jul. 31, 2000, now abandoned.
Application No. 09/628,883 is a continuation-in-part of application No. 08/810,945, filed on Feb. 27, 1997, now Pat. No. 6,103,523.
(60) Provisional application No. 60/214,859, filed on Jun. 28, 2000.

(51) Int. Cl. C12N 5/08; C12N 5/02

(52) U.S. Cl. 435/366; 435/384

(57) ABSTRACT

A novel medium for maintaining and growing pluripotent and germ line competent mammalian stem cells. The medium is conditioned by a fibroblast cell clone that produces leukemia inhibitory factor. The cells are placed in the medium so that, over a period of time, the leukemia inhibitory factor accumulates in the medium. A portion of the medium is then removed, and the quantity removed is replaced with unconditioned medium.

### CROSS REFERENCE TO RELATED APPLICATIONS

This is a continuation-in-part application of U.S. patent application Ser. No. 09/894,913, filed Jun. 28, 2001, which is a continuation-in-part of U.S. patent application Ser. No. 09/628,883, filed Jul. 31, 2000, which is a continuation-in-part application of U.S. patent application Ser. No. 08/810,945, filed Feb. 27, 1997, now U.S. Pat. No. 6,103,523, issued Aug. 14, 2000, and which is incorporated herein by reference, as combined with U.S. Provisional Patent Application Serial No. 60/214,859, filed on Jun. 28, 2000, which is also incorporated herein by reference.

### BACKGROUND OF THE INVENTION

1. Field of the Invention

The present invention relates to improved methods for the derivation of pluripotent embryonic stem (ES) cell lines and their use for germ line transmission and for the generation of genetically modified animals.

2. Description of Related Art

Embryonic stem (ES) cell lines are cell lines isolated from the inner cell mass (ICM) of blastocyst-stage embryos which, under specific conditions, can be maintained in culture for many passages, i.e., replating of cells onto new cell culture dishes at regular time intervals, without loss of their pluripotency. They maintain a normal karyotype and when reintroduced into a host blastocyst they can colonize the germ line. Such cell lines may provide an abundance of pluripotent cells that can be transformed in vitro with DNA, and selected for recombination (homologous or non-homologous) of exogenous DNA into chromosomal DNA, allowing stable incorporation of the desired gene. To date, germ line transmission, i.e., the transmission of the ES genome to the next generation, has however only been achieved with ES cells of certain mouse strains.

Murine embryonic stem cells were first isolated in 1981. Since then, several ES cell lines have been established, and they are now widely and successfully used for the introduction of targeted mutations or other genetic alterations into the mouse genome. Most of the germ line competent mouse ES cell lines that are in current use have been obtained in the 129 strain, and the remainder in a few other inbred strains (C57B16 and crosses with C57B16). Furthermore, ES cell lines are at best obtained in 30% of explanted blastocysts, even in the 129 strain, and success rates of around 10% appear to be closer to the norm. ES cell lines can be transformed in vitro with DNA and selected for recombination (homologous or non-homologous) of exogenous DNA into chromosomal DNA, allowing stable incorporation of the desired gene. Because the genetic background may be important in some phenotypes, ES cell lines from other inbred and mutant mouse strains are desirable.

The most commonly used approach to generate chimeric animals is to inject about 10-15 isolated ES cells into the blastocoel of a host blastocyst and to allow the cells to mix with the cells of the inner cell mass. The resultant chimeric blastocysts are then transferred to recipients for rearing. Alternatively, diploid aggregation using very early (8-16 cell) stage embryos and tetraploid aggregation can be used as hosts for ES cells. Briefly, ES cells are ‘sandwiched’ between early stage embryos devoid of their zona pellucida, cultured overnight and implanted into a foster mother. This technique can be performed under conditions yielding either chimeric or totally ES cell-derived offspring.

Although ES cell culture and chimera production have been technically improved over the years, the pluripotency of the ES cells is still often reduced after several passages, whereas completely ES cell-derived fetuses seem to have a markedly reduced survival after birth. R1 ES cell lines derived from early passages with electrofusion derived tetraploid embryos have been used to form aggregates and obtained mice which were entirely derived from ES cells. However, the R1 ES cells lost their totipotency upon extended culture in vitro, because no animal survived to adulthood from ES cells obtained from later than 14 passages. Moreover, even when early passage cells were used, many ES-tetraploid aggregates died before developing to term. Only 3.8% of transferred aggregates survived after caesarian section. The goal to obtain viable ES mice using later passage ES cells was not reached and the production of ES cell derived mice using genetically modified ES cells did not seem to be possible.

Presumptive pluripotential ES cells have been isolated from a number of species other than mice, including hamster, pig, sheep, cattle, mink, rat, primate, human, chicken, marmoset, medakafish and man. In only a few instances (pig, chicken, medakafish) have these cell lines given rise to chimeras when reintroduced into blastocysts.

The isolation of pluripotential ES cell lines from preimplantation rabbit blastocysts has been reported. These ES lines were found to give rise to differentiated cell types representative of all three germ layers (pluripotential by in vitro criteria). Recently these ES lines from the Dutch Belted strain were shown to be also capable of generating overt coat color chimeras following injection into recipient New Zealand White blastocysts, demonstrating that the cells were pluripotential by in vivo criteria as well. However no germ line transmission has been achieved. Additional experiments showed that the low frequency of chimera formation and absence of germ line transmission probably was due to the loss of pluripotency of the ES cell line upon high passage number.

ES cells are maintained in an undifferentiated state by the presence of feeder layers producing various factor(s) that prevent the cells from differentiating. It has been shown that several cytokines are responsible for this effect: DLA/LIF; differentiation inhibitory activity/leukemia inhibiting factor; interleukin-6 in combination with soluble interleukin-6 receptor; interleukin-11; oncostatin M; ciliary neurotrophic factor; and cardiotrophin. It is now possible to establish and maintain ES cells in culture in the absence of feeder cells but in the presence of such factors. In species other than the mouse, ES cell technology is still under development and there are no published data reporting germ line transmission in any species than mouse.

Advances in recombinant DNA technology over the last decade have greatly facilitated the isolation and manipulation of genes to the point where any conceivable novel construct can be engineered, such as by fusing the promoter of one gene to the coding sequence of another, or by site-directed mutagenesis. Likewise, advances in embryo manipulation have facilitated the transfer of these novel exogenous genes into endogenous chromosomal DNA, generating transgenic animals. Transgenic animals can be generated either by injection of DNA into the pronucleus of zygotes; by introduction of (genetically manipulated) pluripotent embryonic stem (ES) cells into host “embryos”; and, more recently, by nuclear transfer with stably transfected somatic donor cells into enucleated oocytes.

Injection of DNA into pronuclear zygotes generates animals that over- or underexpress specific gene products but integration and expression of the transgene is poorly controlled. Position effects, depending on the site of integration, can have major consequences on the expression of the transgene, including loss of cell specificity, inappropriately high copy number or complete silencing of the transgene. This is of great concern, especially in non-murine transgenesis where the required investment is very high.

Specific targeting of genes via homologous recombination in embryonic stem cells generates mice with inactivated or mutated genes and, more recently, with genes that can be conditionally or tissue-specifically manipulated. Homologous recombination is based on the observation that when a recombinant DNA fragment (the “targeting” DNA) contains sufficient sequence homology with a specific wild-type gene, recombination between the homologous fragments exchanges the wild-type gene with the recombinant targeting DNA. By replacing genomic sequences encoding functionally important protein structures with a selectable marker, such as the neomycin resistance (neo) gene, inactivation of the desired gene is achieved. Incorporation of the marker in the targeting vector permits the isolation of ES cell clones that have stably integrated the targeting vector in their genome by positive selection for resistance. Appropriate Southern blot analysis of genomic DNA from resistant ES cell clones allows the distinction between homologous recombination (rare) and random integration (frequent). Clones of ES cells in which the natural gene is replaced by the inactivated hybrid are selected in vitro, and these selected clones are introduced into normal embryos which are reimplanted. This process generates chimeric animals, which are selected for germ line transmission of the inactivated gene. Through breeding and selection, transgenic animals are then generated which are deficient in the targeted (“knocked-out”) gene. Thus far, gene targeting experiments have been almost exclusively used to inactivate genes in mice.

However, human diseases frequently arise from more subtle gene mutations or from disturbed gene expression in specific tissues at specific times. Therefore, “second generation” gene targeting technology attempts to develop targeted gene mutation; tissue-specific gene targeting; conditional gene targeting; and gene targeting in other species. Targeted gene mutation and tissue-specific gene targeting can be performed using the Cre/loxP system. By flanking target sequences with loxP sites, which are the recognition sequences for the phage Cre-recombinase, it is possible to delete, invert or introduce these target sequences either in vitro or in vivo.

An alternative route towards reinstating the ES genome in the germ line is by means of nuclear transfer, such as in the generation of viable sheep zygotes by fusing individual inner cell mass cells with enucleated oocytes. When applied to ES cells, this route will ensure that all the cells in the offspring, including the germ cells, are of the ES cell genotype. Nuclear transfer is achieved by electrofusing a karyoplast with a surgically enucleated oocyte (cytoplast) derived from in vivo or in vitro sources, but the overall success of this process is only 6-7 lambs or calves born per 100 karyoplasts (blastomeres) fused. This low efficiency is offset by the abundance of ES cells available from a given cell line and the unlimited supply of oocytes from abattoir sources. Recently lambs were born after cells derived from sheep embryos, which had been cultured for 6 to 13 passages, were induced to quiesce by serum starvation before transfer of their nuclei into enucleated oocytes.

Transgenic animals could be useful for the study of the biological effects of identified genes; for the pharmaceutical production of therapeutic gene products; for the generation of “improved” livestock, etc. The larger mammals in which transgene techniques might be cost-effectively applied are limited, depending principally on their gestation period, age to sexual maturity, litter size and maintenance costs. Compared to other larger mammals, rabbits are relatively inexpensive to breed and mate. They have a short gestation period (28-33 days) and reach sexual maturity after 7-9 months. Homozygous transgenic rabbits can be expected after 19 months. In addition, the females are induced ovulators (the mating itself induces ovulation) and the conditions for superovulation are well-known. On average, 20-40 zygotes or blastocysts can be collected per rabbit, limiting the number of donors needed. Reimplantation efficiency of blastocysts resulting in live offspring amounts up to 30-50%, also lowering the number of recipient does needed. These observations make the rabbit an interesting and practically feasible larger animal that can be used as a model to study the effect of targeted mutations and (over) expression of genes.

### SUMMARY OF THE INVENTION

The present invention is directed to a novel medium for maintaining and growing pluripotent and germ line competent mammalian stem cells, especially germ-line competent mammalian embryonic (ES) stem cell line. Pluripotent stem cells can give rise to cells from all three embryonic germ layers: mesoderm, endoderm and ectoderm. Stem cells that may be maintained and grown in this medium are not limited to embryonic stem cells; for instance, stem cells derived from primordial germ cells may be integrated in diploid or tetraploid aggregates. The medium is conditioned by a fibroblast cell clone that produces leukemia inhibitory factor. The cells are placed in the medium so that, over a period of time, the leukemia inhibitory factor accumulates in the medium. A portion of the medium is then removed, and the quantity removed is replaced with unconditioned medium. The accumulation and removal procedures are repeated.

Suitable mammalian cell culture media that may be included in the medium of the present invention includes Phosphate Buffered Saline (PBS); Dulbecco's Modified Eagle Media (D-MEM); Iscove's Modified Media; Dulbecco's Media; McCoy's 5A Media; Minimum Essential Media Eagle (MEM); RPMI Media 1640; Medium 199; MCDB Medium; RPMI; Glasgow Minimum Essential Media (GMEM); DMEMIF-12 Media; Hams F-10 Nutrient Mixture; Lebovitz's L-15 Media; CMRL Media; BGJb Medium; Basal Medium Eagle (BME); Brinster's BMOC-3 Medium; Williams Media E; McCoy's Media; high glucose DMEM; and adaptations thereof.

The medium of the invention typically contains an animal serum or an animal serum replacement. Suitable animal sera include fetal animal serum or newborn animal serum. Fetal bovine serum (FBS), preferably in a dialyzed gamma-irradiated or heat-inactivated form, may be used in this application. Serum derived from sheep, pigs, chickens and horses may also be used. Other reagents typically present include inorganic salts, amino acids, vitamins and sugars in concentrations suitable to culture stem cells.

Other components found to be useful in the medium of the present invention include reducing agents, such as 2-mercaptoethanol or Microhydrin. The reagent 2-mercaptoethanol increases the viability and plating efficiencies of stem cells, as well as serving as a reducing agent. Reagents with antimycotic or antibiotic abilities usable in the medium include penicillin, streptomycin and gentamycin. L-glutamine, ethylene glycol-bis(beta-aminoethyl ether) N,N,N′,N′-tetraacetate (EGTA), insulin and albumin may also be added; the use of albumin is preferred when animal serum replacements are included in the medium.

### BRIEF DESCRIPTION OF THE DRAWING(S)

FIG. 1 is a nucleotide and amino acid sequence listing for rabbit LIF cDNA.

### DESCRIPTION OF THE PREFERRED EMBODIMENT(S)

The invention will be illustrated in the following examples that are not intended to limit the scope of the invention. Based on the present invention, several variants and improvements will be obvious to those skilled in the art.

### EXAMPLE I

### Production of Improved ES Cell Medium which Maintains Embryonic Stem (ES) Cells Undifferentiated

Phage plaques representing a “Sau 3A-partial” rabbit genomic library were grown at a density of 300,000 plaques per 24×24 cm dish and transferred to nitrocellulose in duplicate. This rabbit genomic lamba DASH II library (Stratagene, #955950) was screened with a 1200 bp murine LIF cDNA probe. After hybridization overnight at 42° C., the membrane was washed twice at room temperature for 20 min with 0.5 SSC & 0.5 SDS, for 45 min at 55° C., and for 30 min at 59° C. with 0.2 SSC & 0.5 SDS and then autoradiographed. Plaques positive on duplicate filters were rescreened at lower density. One clone was subjected to sequence analysis and identified as encoding the rabbit LIF protein. A 2.9 kb BamHI fragment containing the complete rabbit LIF genomic DNA was then inserted into an expression cassette with the PGK promoter and the bovine poly A sequence.

Permanent expression of the rabbit LIF gene was achieved in immortalized rabbit fibroblast cells (Rab9 #19 fibroblasts purchased from ATCC, Manassas, Va., USA) by cotransfection of the LIF expression cassette with a cassette encoding for neomycin resistance. The cotransfection was realized with 10 consecutive pulses (99 μsec, 2.5 kV/cm, direct current, BTX electro cell manipulator ECM 200, San Diego, Calif., USA), 5 μg of BgIII & XhoI fragment (4.4 kb) from the neomycine resistance cassette and 15 μg of a HindIII & NotI fragment of the LIF expression cassette. Dulbecco's PBS was used as an electroporation buffer.

The neomycin resistance cassette comprised the PGK promoter (0.5 kb)+n-galineo (3.6 kb)+bovine poly A (325 bp) in the pSP72 vector (2.4 kb). N-galineo was designed and constructed by R. Moreadith as a fusion between the nuclear localizing form of β-galactosidase in frame with neomycin.

The rabbit LIF expression cassette comprised the PGK promoter (0.5 kb)+a BamHI fragment of 2.9 kb containing the rabbit LIF genomic DNA+bovine poly A (325 bp) in the pSP72 vector (2.4 kb).

Basic ES cell medium consists of 500 ml DMEM high glucose (cat no. 12430-054), 13 ml penicillin/streptomycin, 13 ml non-essential amino acids, 13 ml glutamine, 6.3 μl μ mercaptoethanol, 70 ml fetal bovine serum, and pH 7.4.

Non-transfected rabbit fibroblast cells did not produce measurable quantities of rabbit LIF (i.e., less than 20 pg/ml/24 hours, when grown on 15 cm dishes with basic ES medium at 39° C. in a humidified atmosphere of 5% CO₂ in air). After transfection, several G418 (200 μg/ml) resistant colonies were isolated, which also produced rabbit LIF (i.e., more than 20 pg/ml/24 hours or up to 30 ng rabbit LIF/ml/24 hours in the medium when grown on 15 cm dishes with 25 ml basic ES medium at 39° C. in a humidified atmosphere of 5% CO in air).

A transfected fibroblast clone (Rab9 #19) was deposited with the Belgian Coordinated Collection of Microorganisms, under accession no. LMBP 5479CB. This clone secreted up to 30 ng rabbit LIF/ml/24 hours in the medium as measured with the Quantikine human LIF immunoassay kit from R&D Systems (Minneapolis, Minn.).

Basic ES cell medium, conditioned by the Rab9 #19 fibroblast cells, was collected for four consecutive days. Each time, the dishes were refreshed with 25 ml of basic ES medium. After four days, each 15 cm dish was split at a ratio of 1 to 7. The first day after splitting the medium was not collected, but discarded.

Improved ES cell medium consists of 450 ml of conditioned basic ES medium (from the mixture of the four collection days), 60 ml of Basic ES cell medium, 10 ml non-essential amino acids, 10 ml glutamine, 2.3 μl β mercaptoethanol, 70 ml fetal calf serum, 0.6 ml bovine insulin, at pH 7.4.

Basic ES cell medium, conditioned by these rabbit LIF producing fibroblast cells (Rab9 #19), maintained R1 murine ES cells undifferentiated for at least five passages on bare culture dishes. Alkaline phosphatase staining and vimentin staining confirmed the undifferentiated character of the R1 ES cells. Murine ES cells growing in Basic ES cell medium, not conditioned by Rab9 #19 fibroblast cells, differentiated within one or two passages.

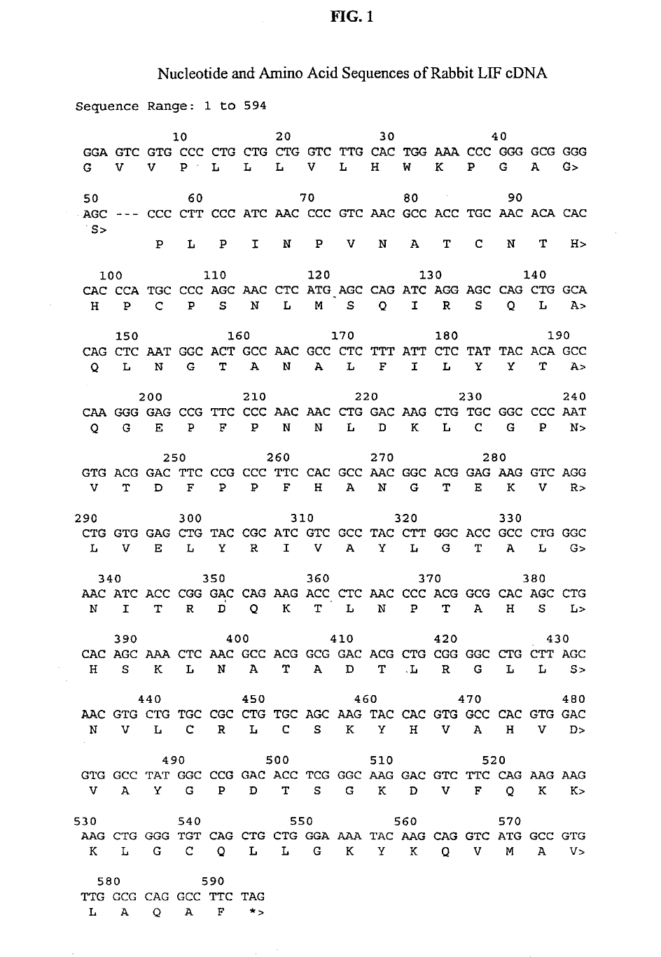

Recombinant rabbit LIF was produced using the methylothropic yeast Pichia pastoris expression system from Invitrogen (Carlsbad, Calif.). The rabbit LIF gene was fused in frame to the α-factor secretion signal in the vector pPICZα. This allows the isolation of the recombinant protein from transformed Pichia pastoris culture supernatant. The nucleotide sequence of the rabbit LIF cDNA, optimized for high expression in Pichia pastoris, is shown in FIG. 1.

Basic ES cell medium to which recombinant rabbit LIF was added at a concentration of 10 ng/ml, maintained murine ES cells undifferentiated for at least 5 passages on bare culture dishes. Alkaline phosphatase staining and vimentin staining confirmed the undifferentiated character of the ES cells. Murine ES cells growing in Basic ES cell medium alone differentiated within one or two passages.

### EXAMPLE II

### Technological Aspects of Mouse Embryonic Stem Cell Derivation, Culture and Generation of Chimeric and ES Cell Derived Animals

Derivation of Murine ES Cells

ES cells were derived from 3.5-4.5-day old blastocyst-stage murine embryos, which were collected and plated individually on a 96-well dish covered with a mitotically arrested mouse embryonic fibroblast feeder monolayer. The blastocysts were allowed to attach to the monolayer, and refed every day with Improved ES cell medium (see Example 1). The following commercially available mouse strains were used: 129/SvEvTaconic (Taconic, Germantown, N.Y., USA); C57BL/6NTacfBr (Taconic); BALB/cAnNTacfBr (Taconic); DBA/2NTacfBR (Taconic); C3H/HeNTac-MTVfBe (Taconic); FVB/NTacfBR (Taconic); Tac:(SW)fBR, Swiss Webster (Taconic); 129/SvJ (The Jackson laboratory, Bar Harbor, Me., USA); C57BL/6J-HPRT <B-M3> (The Jackson Laboratory); C57BL/6JOlaHsd (Harlan, Indianapolis, Ind., USA); CBA/CaOlaHsd (Harlan); and DBA/1OlaHsd (Harlan).

After five to six days in culture, the inner cell mass (ICM) outgrowth was selectively removed from the (remaining) trophectoderm and replated after trypsinization with trypsin-EDTA on a 96-well dish with mitomycin arrested murine fibroblasts. Subsequently, the ES cells were gradually plated on larger culture dishes. ES cells proved to remain undifferentiated for more than 20 passages by using Improved ES cell medium.

Fibroblast feeder layers were obtained from murine embryos of 12.5 days post-coitus pregnant mice. The mice were sacrificed, and the uteri collected and placed in a petri dish containing phosphate buffered saline (PBS). The embryos were dissected out of the uterus, and all membranes removed. The embryos were transferred into a new dish containing PBS, the head and all internal organs removed, and the carcasses washed in PBS to remove blood. The carcasses were then minced using 2 insulin syringes into cubes of 2 to 3 mm in diameter, and incubated in Trypsin-EDTA/MEM solution (10/90 v/v) at 4° C. for 2 hours. The suspension was then incubated at 37° C. for 15 min, a single cell suspension made using a 5 ml pipette, and plated at 5×10⁶ cells per 180 mm petri dish in 25 ml Feeder Medium. Feeder Medium consisted of 500 ml Dulbecco's Minimal Essential Medium (DMEM); 10% fetal calf serum (FCS); 13 ml Penicillin/Streptomycin; 13 ml Glutamine; 13 ml Non-Essential Amino Acids; and 2.3 μl β-mercaptoethanol. The medium was changed after 24 hours to remove debris. After two to three days of culture, the fibroblasts reached a confluent monolayer. The plates were then trypsinized, replated on two petri dishes and, when confluent, the cells of a plate were frozen in two vials, kept at −80° C. overnight, and transferred to liquid nitrogen the next day.

Culture of ES Cells

ES cells were grown to subconfluency on mouse embryonic fibroblasts mitotically arrested with mitomycin. Culture dishes were kept at 39° C. in a humidified atmosphere of 5% CO₂ in air. The ES cells were passaged every three to four days onto freshly prepared feeder dishes. The ES cells were fed every day with the Improved ES cell medium.

Blastocyst Injection or Aggregation of ES Cell Clones

The ability of the ES cells to colonize the germ line of a host embryo was tested by injection of these ES cells into host blastocysts, or by their aggregation with morula-stage diploid embryos or 4-celled tetraploid embryos, and implanting these chimeric preimplantation embryos into pseudopregnant foster recipients according to standard procedures. The resulting chimeric offspring were test bred for germ line transmission of the ES cell genome.

In mice, completely ES cell derived embryos can also be generated in a single step via aggregation of the ES cells with tetraploid host embryos. 2-celled embryos were electrically fused and, subsequently, aggregated as 8-celled tetraploid embryos with the ES cells to form chimeric preimplantation embryos. These were then implanted in pseudopregnant recipients and analyzed during different embryonic stages. The ES cells (almost) exclusively contributed to the development of the embryo proper, and the tetraploid cells to that of the extraembryonic membranes. In order to distinguish between the ES and tetraploid cells, morula embryos (used for aggregation) were derived from the ROSA26 strain, which expresses LacZ ubiquitously and throughout the entire development and adulthood.

ES cells of mouse strains with a coat color (C57B1/6J-HPRT #2, DBA/2N #8, DBA/1 Ola # 36) were injected into host blastocysts of albino Swiss Webster mice. ES cells of mouse strains with a white or creamish coat color (Swiss Webster #43, Swiss Webster #44, 129/SvJ #3, 129/SvJ #4, 129/SvJ #7, BALB/c #17, BALB/c #29, and FVB #17) were injected into host blastocysts of black C57BL/6N mice. This allows easy identification of ES cell contribution. All ES cell lines tested resulted in chimeric offspring with germ line capability (see below).

Diploid Aggregation of ES Cell Clones

The diploid aggregation method was executed as follows. Swiss Webster (albino coat color) females were superovulated with pregnant mare serum gonadotropin followed 44-48 hours later by five units human chorionic gonadotropin. The oviducts of superovulated and mated Swiss Webster mice were flushed 2.5 days after copulation to collect late 8-cell stage diploid embryos. All ES cell lines tested were derived from mice strains with a coat color, facilitating identification of chimeric offspring. Zonae pellucidae of these 8-cell stage diploid embryos were removed by treatment with acid Tyrode's buffer. The zona-free embryos were washed and placed in M16 medium. Aggregation was performed between one 8-cell stage diploid embryo and a clump of ES cells. The aggregates were cultured in micro drops of M16 until the blastocyst stage before they were reimplanted into the uterus horns of 2.5-day pseudopregnant Swiss Webster females. Chimeric pups were identified by the presence of a dark (non-albino) color, which originated from an ES cell contribution. The percentage of chimerism (portion of the newborn pup, originating from the ES cells) was visually identified by judging the percentage of dark coat (originating from the ES cells) compared to the white coat (originating from the albino Swiss Webster embryo).

Tetraploid Aggregation of ES Cell Clones

Completely ES cell derived embryos (13) were generated via aggregation of the ES cells with tetraploid host embryos. 2-celled embryos were electrically fused and, subsequently, aggregated as 4-celled tetraploid embryos with the ES cells to form chimeric embryos, which were then implanted in pseudopregnant recipients. The ES cells (almost) exclusively contributed to the development of the embryo proper, and the tetraploid cells to that of the extra embryonic membranes. In order to distinguish between the ES and tetraploid cells, host embryos (used for aggregation) were derived from the ROSA26 strain, which expresses LacZ ubiquitously and throughout the entire development and adulthood. The oviducts of superovulated and mated ROSA26 mice were flushed 36 hours after treatment with human chorionic gonadotropin to collect late 2-cell stage embryos. Electro fusion was carried out to produce tetraploid embryos. The 2-cell stage embryos were placed between two platinum electrodes laid 250 μm apart in 0.2 M mannitol medium in the electrode chamber (3). The two blastomeres were fused by a short electrical pulse (100 V for 100 μsec in 0.3 M mannitol) applied by a pulse-generator (CF; manufactured by Biochemical Laboratory service, Budapest, Hungary). The fused tetraploid embryos were cultured overnight in M16 micro drops under mineral oil in 37° C. in 95% air/5% CO₂. Twenty-four hours after fusion, most of the tetraploid embryos developed to the 4-cell stage. Only these 4-cell-stage embryos were used for aggregation. Zonac pellucidae of these embryos were removed by treatment with acid Tyrode's buffer. ES cell (plated at low density on bare gelatinised dishes, without feeder layer, two days prior to aggregation) were briefly trypsinized to form clumps of loosely connected cells. Clumps of 10-15 ES cells were sandwiched between two tetraploid embryos in aggregation wells. The aggregates were cultured in micro drops of M16 until the blastocyst stage before they were reimplanted into the uterus horns of 2.5-day pseudopregnant Swiss Webster females.

The germ line transmission capacity of our newly derived ES cells were determined at a passage number of 10 or higher.

### EXAMPLE III

### Derivation of Mouse ES Cell Lines and Generation of ES Cell Derived Animals

Results of ES Cell Derivation

Most of the germ line competent murine ES cell line that are in current use have been obtained in the 129 strain. Since the genetic background may be very important in various studies, it was desirable that ES cell lines are established from various inbred and mutant mice strains. Therefore, we have derived ES cells from one outbred and nine different inbred mouse strains (Table 1) and from five gene-targeted mouse strains (cfr. Table II). In the 129 strains, 61% (129/SvEv) and 58% (129/SvJ) of the explanted blastocyst gave rise to an ES cell line.

1. In the C57BL/6 backgrounds, the efficiency of ES cell derivation was above 30%.

2. ES cells with germ line transmission capability were obtained from CBA/CaOlaHsd mice, a strain previously believed to be non-permissive to ES cell derivation (62).

3. 2 out of 37 Balb/c blastocysts give rise to an ES cell line. Both lines transmitted the ES genome through the germ line.

4. A success rate of 11% was obtained in the DBA/1Ola strain. Roach et al. reported in 1991 a success rate of 0.01% in the DBA/1lacJ strain.

5. ES cells were obtained from the DBA/2N, the FVB/N and Swiss Webster strains with efficiencies of 37%, 22% and 7%, respectively. Successful ES cell derivation from these strains has not previously been reported.

6. Improved ES cell medium allowed derivation of ES cells of genetically manipulated mouse strains with high efficiency. With the exception of the ApoE-/-mice (11%), the efficiency of ES cell derivation was consistently above 30%, varying between 35 and 58% (Table II).

Results of Germ Line Transmission Testing After Blastocyst Injection

ES cells of mouse strains with a coat color (C57B1/6J-HPRT<B-M3> #2, DBA/2 #8, DBA/1 Ola # 36) were injected into host blastocysts of albino Swiss Webster mice. ES cells of white mouse strains (Swiss Webster #43, Swiss Webster #44, 129/SvJ #3, 129/SvJ #4, 129/SvJ #7, Balb/C #17 and FVB #17) were injected into host blastocysts of black C57BL/6N mice. This allows an easy identification of ES cell contribution. All ES cell lines tested resulted in chimeric offspring with germ line capability (Table III).

Results of Germ Line Transmission Testing After Diploid Aggregation

The germ line transmission capacity of four different mouse strains was tested after diploid aggregation with 8-celled embryos of the Swiss Webster strain. The Swiss Webster is an albino strain, while all ES cell lines involved in this experiment were derived from mice strains with a coat color, facilitating identification of chimeric offspring. All of the ES cell lines tested by diploid aggregation were able to produce chimeric offspring with germ line transmission capacity (Table IV).

Overall, between 5-15% of all embryos reimplanted after diploid aggregation resulted in live offspring with an ES cell contribution. The percentage of chimerism of all offspring born with an ES cell contribution was very high. All chimeric mice born after diploid aggregation of ES cells from C57BL/6N #25; C57BL/6N #28; C57B1/6J-HPRT #2; and 129SvEv #4 with embryos of the Swiss Webster strain had 100% chimerism. After diploid aggregation with the 129SvEv #7 ES cell line, three out of five chimeric animals born were 100% chimeric for the ES cell line.

Results of Germ Line Transmission Testing After Tetraploid Aggregation

Several of our ES cell lines were tested for their germ line transmission capability after tetraploid aggregation (Table V). Completely ES cell derived embryos were generated in a single step via aggregation of the ES cells with tetraploid Swiss Webster host embryos. 2-celled Swiss Webster embryos were electrically fused and, subsequently, aggregated with the ES cells to form 100% ES derived offspring.

1. Four of the established 129SvEv ES cell lines tested in tetraploid aggregation produced completely ES cell derived offspring after tetraploid aggregation. Between 3 and 30% of the reimplanted embryos produced live offspring. Tetraploid aggregation of ES cell line #7 of the 129SvEv strain at passage 17 was carried out with Rosa 26 tetraploid blastomeres and 13 and 10 aggregates were transferred to two foster mothers yielding three and four live offspring, respectively. All seven offspring were totally ES cell derived and fertile having produced one to four litters comprising of 11 to 40 pups.

2. Seven pups (12% of all reimplanted embryos) were born after tetraploid aggregation of a selected C57BL/6 ES cell line at passage 12 with Rosa 26 tetraploid blastomeres. Two males, randomly selected out of the seven, showed germ line transmission.

3. The improved ES cell culture medium and derivation conditions for murine ES allowed to derive ES cells with germ line transmission capability from CBA/CaOlaHsd mice, a strain previously believed to be non-permissive to ES cell derivation, after tetraploid aggregation.

4. ES cells (TF^{+/+}, TF^{+/-}, TF^{-/-}) were derived from embryos of heterozygous tissue factor (TF) deficient breeding pairs as described above, passaged, and used for tetraploid aggregation. Of 14 transferred aggregates, two pups were born with the correct TF^{+/-} genotype. Both were germ line transmitting.

With the availability of these ES cells, it will be possible to induce mutations in the genetic background of choice and to analyze the induced mutation without time-consuming inbreeding. Furthermore, these ES cells may be used to generate transgenic “gain-of-function” mice since it is highly inefficient and expensive to produce transgenic mice via pronuclear microinjection in backgrounds other than FVB and C57BL/16.

### EXAMPLE IV

### Larger Scale Production and Evaluation of Improved ES Cell Medium

Larger Scale Production of Rab9 #19 Conditioned Medium

The cryopreserved Rab9 #19 cells (107 cells) were thawed and seeded in two T175 flasks. Upon confluence, the cells were passaged in a 1200 cm² cell factory at a density of 25 000 cells/cm². Upon confluence, the cells were harvested and seeded in a 3L bioreactor containing 1L of Improved ES cell medium and 2.47 g of cytodex 3 at a density of 15 000 cells/cm².

The bioreactor (Applicon, 3L) was equipped with a marine-type impeller and a perfusion system. Aeration was performed through a microsparger. The pH was continuously monitored and maintained at 7.4 by addition of 1N NaOH.

The suspension was sampled daily to monitor the cell growth and LIF concentration. When the LIF concentration reached values between 15 and 20 ng/ml (approximately at day 3-4), the perfusion was initiated at a rate of about 0.5 L/day. The culture was maintained for 30 days. The perfusion rate was adapted over the life of the culture to result in a LIF concentration of 18-20 ng/ml. The perfusate was collected at 4° C. by 3-day pool.

The Improved ES cell medium was subsequently constituted as follows. To each liter collected (3-day pool) perfilsate; 80 ml fetal bovine serum; 17 ml non-essential amino acids; 5 μl β mercaptoethanol; 1.25 ml insulin; 80 to 130 ml basic ES cell medium (to adjust the LIF to a final concentration of 14 to 15 ng/ml) was added. The Improved ES cell medium was filtered on 0.22 micron cellulose acetate filters and frozen at −80° C. Upon usage, 20 ml glutamine was added per liter Improved ES cell medium.

Derivation of Morse ES Cell Lines from Two Inbred Mouse Strains with Improved ES Cell Medium Produced on a Larger Scale

The quality of this Improved ES cell medium (produced on a larger scale) was tested by evaluating its potential to allow the establisment of ES cell lines from C57BL/6NTacfBr (Taconic, Germantown, N.Y., USA) and FVB/NTacfBR (Taconic) mouse. When 3.5-day old blastocysts were collected from C57BL/6N and FVB/N mouse, and ES cells were derived according to the procedures described earlier, respectively, 58% and 50% of the blastocysts gave rise to an ES cell line.

### EXAMPLE V

### Derivation of Rabbit ES Cell Lines with Germ Line Transmission Capability

It was earlier reported that ES lines from the Dutch Belted strain were capable of generating overt coat-color chimeras following injection into recipient New Zealand White blastocysts, demonstrating that the cells were pluripotential by in vivo criteria. However, no germ line transmission was obtained. Since then, the ES cell culture conditions were further improved.

For the derivation of rabbit ES cells, the following growth factors and cytokines were added to the Improved ES cell medium described in Example I, to concentrations of 10 ng/ml; human Interleukin 11; human Oncostatin M; human Ciliary Neurotrophic factor; human stem cell factor; and basic fibroblast growth factor. This medium which will be called hereafter SM-ESI medium allowed the derivation of several rabbit ES cell lines, consisting of more than 80% undifferentiated alkaline phosphatase positive and vimentin negative cells, after 20 passages in vitro.

New Zealand Brown rabbits were obtained from University of Texas (Southwestern, Dallas, Tex.) (Dr. R. W. Moreadith), where they had been generated by pronuclear injection of New Zealand White zygotes with the mouse tyrosinase gene under control of its own endogenous promotor. Homozygous New Zealand Brown (NZBB) rabbits have a brown coat color and pigmented eyes, but are otherwise genetically identical to New Zealand White rabbits.

4.5-5.5-day old blastocysts were flushed from the uterine horns of does. The mucin coat and/or zona pellucida were removed using acidified PBS and pronase. The inner cell mass was prepared out of the surrounding trophectoderm cells, and placed individually on 96-well culture dishes, plated with mitomycin arrested mouse embryonic or rabbit embryonic fibroblast.

The inner cell masses were allowed to attach to the monolayer, and refed every day with SM-ES1 medium. After 5-6 days in culture, the ICM outgrowth was selectively removed from the (remaining) trophectoderm and replated again after trypsinization with a selective trypsinization medium consisting of 0.1% collagenase, 1% chicken serum and 0.03% trypsin-EDTA in phosphate buffered saline on a 96-well dish with mitomycin arrested murine or rabbit fibroblasts. Subsequently, the ES cells were gradually plated on larger culture dishes. The pluripotential character of newly derived ES cell lines was checked by alkaline phosphatase staining and by their capacity to give rise to cells representative of the three germ layers (ecto-, meso- and endoderm) under conditions that promote differentiation.

NZB ES were derived from blastocysts resulting from the mating of NZW females with NZBB males. NZBB ES cells were derived from the mating of NZBB females with NZBB males. In the first case, the tyrosinase transgene is only present on one chromosome, in the second case on both chromosomes.

To test to ability of the newly derived ES cells to produce chimeras, they were injected in NZW blastocysts. NZW blastocyst injections with NZB or NZBB ES cells were performed on a Zeiss inverted microscope with differential optics. Micromanipulations were performed with Narashige micromanipulators similar to the procedures normally used in the mouse.

The new ES medium and ES cell derivation methods allowed the derivation of eight ES cell lines from 17 NZBB×NZW blastocysts and 29 ES cell lines from 45 NZBB×NZBB blastocysts. The pluripotential character of newly derived ES cell lines was checked by alkaline phosphatase staining and vimentin staining. After 20 passages, more than 90% of ES cells were alkaline phosphatase positive and vimentin negative.

In a first experiment, two different ES cell lines (NZBB68 and NZB7) were tested and both produced chimeras after blastocyst injection (Table VI). After birth, the female offspring was sacrificed, and the chimerism was checked by Southern blotting for the presence of the murine tyrosinase gene. With the NZBB68 ES cell line, two out of six offspring rabbits were chimeric with transgene contribution to heart and kidney tissue. With the NZB7 ES cell line, one out of three females was chimeric with transgene contribution to muscle tissue. The male offspring (three with the NZBB68 and two with the NZB7 ES cell line) was tested for germ line transmission, but did not transmit the transgene.

A second experiment with blastocyst injection of NZB7 ES cells confirmed the capacity of this ES cell line to generate chimeric offspring. 20 NZW blastocysts were injected with a cluster of NZB7 ES cells (n=20). Five rabbits were born, of which four females were used to screen for ES cell contribution by PCR. ES cell contribution was demonstrated by the presence of a 380 bp fragment. All four animals had an ES cell contribution in several organs. Rabbit 1 was chimeric in the lungs, the skin and the brain. Rabbit 2 had ES cell contribution to the lungs, the liver, the eyes and the skin. Rabbit 3 did not only have ES cell contribution to the lungs, the brains, the skin and the eyes, but also showed an ES cell contribution to the reproductive organ. Rabbit 4 was chimeric in the skin and in the spleen (data not shown). The specificity of the resulting PCR-fragments (380 bp) was confirmed by nucleic acid sequencing. The sequence of the 380 bp PCR fragments showed a 100% homology to nucleotides 8-388 of the murine tyrosinase cDNA. The murine tyrosinase is naturally not present in NZW rabbits and can only originate from NZB ES cells. A skin sample of the male rabbit was taken. The chimerism was checked (together with a lung and skin sample of female rabbit 1, and a brain and lung sample of rabbit 3) by Southern blotting for the presence of the murine tyrosinase gene.

Although the coat color of the male was white, an ES contribution was clearly demonstrated (not shown). After reaching sexual maturity, the male was mated with several females to test his germ line transmission capacity. Thus far, two litters were born. The doc of the first litter killed all her offspring after two days but skin samples of the dead kittens were recovered for DNA extraction and screening for ES cell contribution by PCR. ES cell contribution was demonstrated by the presence of a 380 bp fragment. One of the dead kittens screened positive for the presence of the tyrosinase minigene by PCR. The specificity of the resulting PCR-fragment (380 bp) was confirmed by nucleic acids sequencing. All five kittens of the second litter, however, tested negative for the tyrosinase gene.

TABLE I
Establishment of ES Cell Lines from One Outbred (Swiss Webster)
and Nine Inbred Mouse Strains

| | | | Efficiency in % |
|---|---|---|---|
| | No. of | | (No. ES cell lines/ |
| | blastocysts | No. of established | No. blastocyst |
| Strain | cultured | ES cell lines* | cultured) |
| 129/Sv Ev | 18 | 11 | 61 |
| C57BL/6N | 30 | 12 | 40 |
| C57BL/6J-HPRT | 25 | 8 | 32 |
| Balb/c | 37 | 2 | 5 |
| CBA/CaOla | 12 | 8 | 66 |
| 129/SvJ | 12 | 7 | 58 |
| DBA/2N | 16 | 6 | 37 |
| DBA/1Ola | 36 | 4 | 11 |
| FVB/N | 18 | 4 | 22 |
| Swiss Webster | 85 | 6 | 7 |

*All ES cell lines have sustained 10 or more passages.

TABLE II
Establishment of ES Cell Lines from Six Genetically Manipulated
(Knock Out) Mouse Strains

| | | | Efficiency in % |
|---|---|---|---|
| | | No. of | (No. ES cell |
| | No. of | estab- | lines/No. |
| | blastocysts | lished ES | blastocyst |
| Strain | cultured | cell lines | cultured) |
| Cenos −/− × Cenos −/− in | 12 | 6 | 50 |
| 129Sv × C57BL/6 | | | |
| Survivin +/− × Survivin +/− in | 100 | 44 | 44 |
| 129SvJ × 129SvPas | | | |
| HIF1α +/− × HIF1α +/− in | 57 | 20 | 35 |
| Swiss Webster × (129SvJ × | | | |
| 129SvPas) | | | |
| ApoE −/− × ApoE −/− in 100% | 18 | 2 | 11 |
| C57BL/6 | | | |
| TF +/− × TF +/− 87.5% | 45 | 26 | 58 |
| C57BL/6 × (12.5% 129Sv JX | | | |
| 129SvPas) | | | |

TABLE III
Production of Chimaeric Mice with Germ Line Transmission Capability
After Blastocyst Injection with Established ES Cells

| | Passage | Blastocysts | Animals | |
|---|---|---|---|---|
| Strain & ES cell line No. | No. | injected | born | Chimaeras |
| C57B1/6J-HPRT<B-M3> | 12 | 60 | 40 | 23 |
| (#2) | | | | |
| DBA/2 #8 | 15 | 60 | 17 | 4 |
| Swiss Webster #43 | 12 | 36 | 9 | 5 |
| | 13 | 37 | 8 | 3 |
| | 14 | 47 | 3 | 1 |
| | 15 | 33 | 9 | 2 |
| Swiss Webster #44 | 14 | 15 | 7 | 4 |
| 129/SvJ #3 | 10 | 20 | 4 | 2 |
| | 14 | 33 | 11 | 8 |
| 129/SvJ #4 | 11 | 33 | 6 | 2 |
| | 15 | 32 | 3 | 1 |
| 129/SvJ #7 | 16 | 30 | 7 | 6 |
| Balb/c ANTacfBr #17 | 17 | 36 | 16 | 11 |
| FVB #17 | 10 | 50 | 18 | 3 |
| | 16 | 34 | 12 | 5 |
| DBA/1 Ola #36 | 11 | 36 | 10 | 0 |
| | 21 | 43 | 14 | 9 |

TABLE IV
Production of Chimaeric Mice with Germ Line Transmission Capability
After Diploid Aggregation with Established ES Cells

| Strain & | Passage | No. embryos | Animals | Chimaeras |
|---|---|---|---|---|
| ES cell line No. | No. | reimplanted | born | (% chimaerism) |
| C57BL/6 #25 | 13 | 99 | 47 | 19 Males (100%) |
| C57BL/6 #28 | 13 | 89 | 40 | 8 Males (100%) |
| C57Bl/6J-HPRT | 13 | 65 | 26 | 5 Males (100%) |
| #2 | 14 | 114 | 28 | 6 Males (100%) |
| 129SvEV #4 | 12 | 59 | 24 | 3 Males (100%) |
| | | | | 1 Female (100%) |
| 129SvEV #7 | 13 | 32 | 12 | 3 Males (100%) |
| | | | | 1 Male (60%) |
| | | | | 1 Female (10%) |
| 129Sv Ev #11 | 12 | 98 | 16 | 8 Males (100%) |
| 129SvEv #17 | 12 + 13 | 82 | 10 | 5 Males (100%) |
| CBA #4 | 12 | 80 | 23 | 4 Males (100%) |
| | | | | 2 Males (50%) |
| | | | | 1 Female (50%) |
| | | | | 1 Female (40%) |

TABLE V
Production of Completely ES derived Cell Mice with Germ Line
Transmission Capability After Tetraploid Aggregation with Established
ES Cells

| | Passage | No. of embryos | |
|---|---|---|---|
| Strain & ES cell line No. | No. | reimplanted | Animals born* |
| C57BL/6 #25 | 12 | 56 | 7 |
| 129SvEV #4 | 13 | 10 | 1 |
| 129SvEV #7 | 12 | 53 | 11 |
| | 16 | 66 | 10 |
| | 17 | 23 | 7 |
| 129Sv Ev #11 | 12 | 132 | 7 |
| 129SvEv #17 | 12 | 139 | 5 |
| CBA #4 | 11 | 67 | 1 |
| 87.5% C57BL/6 × (12.5% | 10 | 14 | 2 |
| 129SvJ × 129SvPas) TF | | | |
| #9+/− | | | |

*All animals born were 100% chimaeric males.

TABLE VI
Establishment of Germ Line Transmission in Rabbits

| | MALE OFFSPRING | | FEMALE | |
|---|---|---|---|---|
| | | GERMLINE | OFFSPRING | |
| | BORN | TRANSMISSION | BORN | CHIMERA |
| NZBB68 | 3 | neg | 6 | 2 |
| NZB7 | 2 | neg | 3 | 1 |
| | 1 | (1 of 7 pups, pos by PCR) | 4 | 4 |

The invention claimed is:

1. A medium of cell culture reagents, for the maintenance and growth of a pluripotent and/or germ line competent mammalian embryonic (ES) stem cell line, which medium is conditioned by a fibroblast cell clone that produces leukemia inhibitory factor.

2. The medium of claim 1, wherein the LIF producing fibroblast cell clone further comprises immortalized rabbit fibroblasts, and further comprises an animal serum or an animal serum replacement.

3. The medium of claim 1, wherein the cell culture reagents are selected from the group consisting of inorganic salts, amino acids, vitamins and sugars.

4. The medium of claim 2, wherein the serum is a fetal animal serum.

5. The medium of claim 2, wherein the serum is a newborn animal serum.

6. The medium of claim 1, comprising reagents selected from the group consisting of Phosphate Buffered Saline (PBS); Dulbecco's Modified Eagle Media (D-MEM); Iscove's Modified Media; Dulbecco's Media; McCoy's SA Media; Minimum Essential Media Eagle (MEM); RPMI Media 1640; Medium 199; MCDB Medium; RPMI; Glasgow Minimum Essential Media (GMEM); DMEM/F-12 Media; Hams F-10 Nutrient Mixture; Lebovitz's L-15 Media; CMRL Media; BGJb Medium; Basal Medium Eagle (BME); Brinster's BMOC-3 Medium; Williams Media E; and McCoy's Media.

7. The medium of claim 4, wherein the fetal animal serum is fetal bovine serum (FBS).

8. The medium of claim 7, wherein the fetal bovine serum is treated by a treatment selected from the group of dialysis, gamma irradiation or heat inactivation.

9. The medium of claim 1, further comprising a reducing agent.

10. The medium of claim 9, wherein the reducing agent is selected from the group consisting of 2-mercaptoethanol and microhydrin.

11. The medium of claim 1, further comprising an antibiotic.

12. The medium of claim 11, wherein the antibiotic is selected from the group consisting of penicillin, streptomycin and gentamycin.

13. The medium of claim 1, further comprising L-glutamine.

14. The medium of claim 1, further comprising EGTA.

15. The medium of claim 1, further comprising albumin.

16. The medium of claim 4, wherein the serum is derived from an animal selected from the group consisting of sheep, pigs, chickens and horses.

17. The composition of claim 16, wherein the immortalized fibroblasts have been transfected, transformed or infected by a vector overexpressing a LIF gene.

18. The composition of claim 17, wherein the LIF gene is a rabbit LIF gene.

19. The composition of claim 18, wherein the fibroblast cell line used for conditioning is the Rab9 #19 cell line, which has been deposited with the Belgian Coordinated Collection of Microorganisms, under accession number LMBP 5479 CB.

20. A process of culturing mammalian ES stem cells to obtain pluripotent and/or germ line competent ES cells, wherein the culturing of the mammalian ES stem cells is at least partially performed in a composition as claimed in claim 1.

21. The process of claim 20, comprising the steps of:
a) culturing cells of blastocyst stage embryos;
b) culturing isolated inner mass cells; and
c) passaging the inner mass cells periodically in a composition as claimed in claim 1.

22. The process of claim 21, wherein the inner mass cells are periodically passaged for at least eight times.

23. The process of claim 20, further comprising the step of producing transgenic animals.

24. Embryonic stem (ES) cell line with germ line transmission capability.

25. The cell line according to claim 20, which has germ line transmission capability after 11 or more passages.

26. The cell line of claim 24, obtainable by the process of claim 20.

27. The cell line of claim 24, wherein the cell line is a murine cell line.

28. The cell line of claim 27, wherein the cell line has been derived from cells or tissues with 129/SvEv; C57BL/6N; C57BL/6J-HPRT; BALB/c; CBA/CaOla; 129/SvJ; DBA/2n; DBA/1 Ola; C3H/HeN; C57B1; 6Jol1; FVB; or Swiss Webster genetic backgrounds.

29. The cell line of claim 28, which has a germ line transmission capability after 11 or more passages.

30. The cell line of claim 29, wherein the cell line is cultured in a composition as claimed in claim 1 supplemented with cytokines and growth factors.

31. Embryonic stem (ES) cell line of claim 24, characterized by three-dimensional colony formation, positive staining for alkaline phosphatase; and negative staining for cytokeratin 18 and virnentin after more than 10 passages.

32. Embryonic stem (ES) cell line of claim 24, for use in the generation of chimeric or ES cell derived animals.

33. Embryonic stem (ES) cell line of claim 24, alteration by homologous or non-homologous recombination.

34. Embryonic stem (ES) cell line of claim 24, for use in the generation of animals with gene alteration via germ line transmission.

35. The method of using the ES cell line of claim 24, for generation of chimeric animals.

36. The method of using the ES cell line of claim 35, for the generation of chimeric animals following blastocyst injection into recipient blastocysts or embryo aggregation or nuclear transfer.

37. The method of differentiating the ES cell line of claim 24, for the study or isolation of (novel) genes.

38. The method of using the ES cell line of claim 24, for the expression or overexpression of genes.


SEQUENCE LISTING (2 sequences)

SEQ ID NO: 1 - DNA, 591 bp, Oryctolagus cuniculus
  Rabbit Leukaemia Inhibitory Factor (LIF)
ggagtcgtgcccctgctgctggtcttgcactggaaacccggggcggggagcccccttcccatcaaccccgtcaacgccacctgcaacacacaccacccatgccccagcaacctcatgagccagatcaggagccagctggcacagctcaatggcactgccaacgccctctttattctctattacacagcccaaggggagccgttccccaacaacctggacaagctgtgcggccccaatgtgacggacttcccgcccttccacgccaacggcacggagaaggtcaggctggtggagctgtaccgcatcgtcgcctaccttggcaccgccctgggcaacatcacccgggaccagaagaccctcaaccccacggcgcacagcctgcacagcaaactcaacgccacggcggacacgctgcggggcctgcttagcaacgtgctgtgccgcctgtgcagcaagtaccacgtggcccacgtggacgtggcctatggcccggacacctcgggcaaggacgtcttccagaagaagaagctggggtgtcagctgctgggaaaatacaagcaggtcatggccgtgttggcgcaggccttctag

SEQ ID NO: 2 - protein, 196 aa, Oryctolagus cuniculus
  Rabbit Leukaemia Inhibitory Factor (LIF)
GVVPLLLVLHWKPGAGSPLPINPVNATCNTHHPCPSNLMSQIRSQLAQLNGTANALFILYYTAQGEPFPNNLDKLCGPNVTDFPPFHANGTEKVRLVELYRIVAYLGTALGNITRDQKTLNPTAHSLHSKLNATADTLRGLLSNVLCRLCSKYHVAHVDVAYGPDTSGKDVFQKKKLGCQLLGKYKQVMAVLAQAF